(19) United States
(12) Patent Application Publication
(10) Pub. No.: US 2023/0233860 A1
(43) Pub. Date: Jul. 27, 2023

(54) CLOSED-LOOP PERIPHERAL NERVE STIMULATION FOR RESTORATION IN CHRONIC PAIN

(71) Applicant: THE JOHNS HOPKINS UNIVERSITY, Baltimore, MD (US)

(72) Inventors: Sridevi V. Sarma, McLean, VA (US); Yun Guan, Baltimore, MD (US); Claire Zurn, Baltimore, MD (US); Christine Beauchene, Chantilly, VA (US)

(73) Assignee: THE JOHNS HOPKINS UNIVERSITY, Baltimore, MD (US)

(21) Appl. No.: 18/002,119

(22) PCT Filed: Jun. 23, 2021
(86) PCT No.: PCT/US2021/038705

§ 371 Date: Dec. 16, 2022

Related U.S. Application Data
(60) Provisional application No. 63/043,431, filed on Jun. 24, 2020.

(51) Int. Cl. A61N 1/36; A61N 1/05

(52) U.S. Cl. CPC A61N 1/36139; A61N 1/36071; A61N 1/0551

(57) ABSTRACT

A closed-loop implantable neurostimulator system for mitigating chronic pain, the closed-loop implantable neurostimulator system including a neuromodulation device comprising one or more electrodes configured to measure a physiological signal of a subject and deliver an electrical stimulation signal to a target area in the subject and a controller, in communication with the one or more electrodes, comprising a processor and a computer-readable memory storing a trained healthy computer model, the controller configured to analyze the physiological signal that is measured using the trained healthy computer model to identify a corrective electrical stimulation signal that, when delivered by the one or more electrodes to the target area, reduces pathological neuronal events in the target area while preserving acute pain response.

### CROSS-REFERENCE TO RELATED APPLICATIONS

This application claims priority to U.S. provisional patent application No. 63/043,431 filed on Jun. 24, 2020, which is hereby incorporated by reference in its entirety.

### GOVERNMENT RIGHTS

This invention was made with government support under grant/contract numbers R01 AT009401-01 awarded by the National Institutes of Health (NIH)/Department of Health and Human Services (DHHS) and T32 NS070201 awarded by the National Institutes of Health (NIH)/National Institute for Neurological Disorders and Strokes (NINDS). The government has certain right in the invention.

### FIELD

The present teachings generally relate closed-loop peripheral nerve stimulation for restoration in chronic pain.

### BACKGROUND

Acute pain is an early-warning physiological signal triggered in the nervous system, which is essential for detecting and minimizing contact with painful, or noxious, stimuli. However, the body's ability to process pain is fragile because inflammation, nerve injury, and malfunction of the nervous system may divert its function, which can result in a debilitating disease known as chronic pain. Chronic pain is defined as pain lasting beyond the time required to heal an injury or longer than 12 weeks. Currently, nearly 100 million adults in the US are affected by chronic pain, which is more than the total affected by heart disease, cancer, and diabetes combined. Each year, chronic pain costs $560-635 billion in medical expenses and lost productivity. Those suffering from chronic pain report some of the lowest quality-of-life levels among people with major illnesses because it is often associated with moderate to severe depression, fatigue, anxiety, trouble sleeping, and changes in appetite. All of these factors can make it difficult to maintain healthy relationships with family and friends, and employment

Chronic pain is primarily treated with drugs, which can have significant negative side effects. A promising alternative therapy is electrical peripheral nerve stimulation (PNS) therapy. However, it has been associated with suboptimal efficacy since its modulation mechanisms are unclear and the current therapies are primarily open-loop (i.e. manually adjusting the stimulation parameters). The programming of the stimulation (selective delivery of pulse width, frequency, and amplitude) is often performed by trial-and-error, and is kept constant (i.e., is open-loop) between programming sessions. Closed-loop stimulation, in contrast, adapts over time to the system's needs by automatically adjusting the parameters in response to a measured pain signal in the body. Closed-loop approaches in engineering systems are often designed based on models that mathematically characterize how a system responds to an actuation signal. Conventional closed-loop approaches for reducing pain, however, are model-free and simply wait for measured pain activity in the spinal cord to cross a threshold before activating suppressive stimulation. This acts as a local anesthetic, suppressing pathological pain, but unfortunately, it also blocks acute pain that alerts the body to damaging stimuli.

### SUMMARY

In accordance with examples of the present disclosure, a closed-loop implantable neurostimulator system for mitigating chronic pain is disclosed. The closed-loop implantable neurostimulator system comprises a neuromodulation device comprising one or more electrodes configured to measure a physiological signal of a subject and deliver an electrical stimulation signal to a target area in the subject and a controller, in communication with the one or more electrodes, comprising a processor and a computer-readable memory storing a trained healthy computer model, the controller configured to analyze the physiological signal that is measured using the trained healthy computer model to identify a corrective electrical stimulation signal that, when delivered by the one or more electrodes to the target area, reduces pathological neuronal events in the target area while preserving acute pain response.

Various additional feature of the closed-loop implantable neurostimulator system can including the following. The target area comprises a dorsal horn and/or the ventral posterolateral nucleus (VPL) of the thalamus and/or the peripheral nerves. The target area comprises a superficial lamina of the dorsal horn or a deep lamina of the dorsal horn or VPL Thalamus or the peripheral nerves. The corrective electrical stimulation signal causes at least one of neuronal inhibition in the target area, neuronal excitation in the target area, and no induced change to neuronal activity in the target area. The closed-loop implantable neurostimulator system further comprises a pulse generator that generates the corrective electrical stimulation signal under the direction of the controller. The closed-loop implantable neurostimulator system further comprises a power source that powers the controller and the pulse generator. The closed-loop implantable neurostimulator system further comprises an insulated lead coupling of one or more electrodes to the controller. The controller further identifies the corrective electrical stimulation signal by minimizing a difference between a computer model representing the response to stimuli of a pathological dorsal horn system and a computer model representing the response to stimuli of a healthy dorsal horn system.

In accordance with examples of the present disclosure, a computer-implemented method for controlling an implantable neurostimulator system for mitigating chronic pain is disclosed. The computer-implemented method comprises measuring, by using one or more electrodes, a physiological signal at a target area in a subject; analyzing, using a controller in communication with the one or more electrodes, the physiological signal that is measured using a trained healthy computer model to identify a corrective electrical stimulation signal that, when delivered by the one or more electrodes to the target area, reduces pathological neuronal events in the target area while preserving acute pain response; and delivering an electrical stimulation signal to the target area of the dorsal horn in the subject.

Various additional features can be provided by the computer-implemented method for controlling an implantable neurostimulator system for mitigating chronic pain including the following. The target area comprises a dorsal horn, VPL Thalamus, or peripheral nerves. The target area comprises a superficial lamina of the dorsal horn or a deep lamina of the dorsal horn, VPL Thalamus, or peripheral nerves. The corrective electrical stimulation signal causes at least one of neuronal inhibition in the target area, neuronal excitation in the target area, and no induced change to neuronal activity in the target area. The computer-implemented method further comprises generating, by a pulse generator, the corrective electrical stimulation signal under the direction of the controller. The computer-implemented method further comprising identifying, by the controller, the corrective electrical stimulation signal by minimizing the difference between a computer model representing the response to stimuli of a pathological dorsal horn system and a computer model representing the response to stimuli of a healthy dorsal horn system with the healthy computer model.

In accordance with examples of the present disclosure, a closed-loop implantable neurostimulator system for mitigating chronic pain is disclosed. The closed-loop implantable neurostimulator system comprises a controller, in communication with one or more electrodes, comprising a processor and a computer-readable memory storing a trained healthy computer model, the controller configured to analyze the physiological signal that is measured using the trained healthy computer model to identify a corrective electrical stimulation signal that, when delivered by the one or more electrodes to the target area, reduces pathological neuronal events in the target area while preserving acute pain response.

Various additional features can be provided by the closed-loop implantable neurostimulator system including the following. The target area comprises a dorsal horn and/or VPL Thalamus, and/or peripheral nerves. The target area comprises a superficial lamina of the dorsal horn or a deep lamina of the dorsal horn and/or VPL Thalamus and/or peripheral nerves. The corrective electrical stimulation signal causes at least one of neuronal inhibition in the target area, neuronal excitation in the target area, and no induced change to neuronal activity in the target area.

### BRIEF DESCRIPTION OF THE DRAWINGS

The accompanying drawings, which are incorporated in, and constitute a part of this specification, illustrate implementations of the present teachings and, together with the description, serve to explain the principles of the disclosure. In the figures:

FIG. 1 shows an illustration of the dorsal horn in the spinal cord. Afferent fibers enter the superficial lamina and the WDR neuron (W) in the deep lamina projects the impulse to the brain.

FIG. 2 shows a biophysical representation of the dorsal horn where the black triangles represent the recording microelectrodes and the WDR neuron (W) projects the impulse to the brain.

FIG. 3A and FIG. 3B show a comparison of the naive and nerve-injured rat responses from the superficial lamina (LFP recordings) in FIG. 3A and deep lamina (WDR firing rate) in FIG. 3B. The solid line and shaded area indicates the mean and standard deviation over the ten trials, respectively.

FIG. 4 shows a generalized block diagram representation of the dorsal horn.

FIG. 5 shows the standard H∞ optimization problem.

FIG. 6 shows a closed-loop system using the controllers, K₁, K₂, and K₃, to drive the injured model, G_{I}, to match the naive model, G_{N}.

FIG. 7A and FIG. 7B show a comparison of the recorded and estimated LFP responses from the optimized transfer functions for the naive and nerve-injured rats, respectively.

FIG. 8 shows a comparison of the bode diagrams for the fitted delay and superficial lamina models for the naive and nerve-injured rats.

FIG. 9A and FIG. 9B show comparison of the recorded and estimated WDR firing rates from the optimized transfer function for the naive and nerve-injured rats, respectively.

FIG. 10 shows a comparison of the bode diagrams for the fitted deep lamina models for the naïve and nerve injured rats.

FIG. 11A shows a comparison of the measured WDR firing rate response in the naïve rat and the closed-loop injured model response.

FIG. 11B shows a resulting control action, U(t), which acts as the input to the injured model.

FIG. 12A shows a comparison of bode diagrams for the naïve, injured, and the closed-loop models.

FIG. 12B shows a bode diagram for the controller.

FIGS. 13A-13D show a comparison of metrics describing the firing rate responses in the naive rat, injured rat, and closed-loop injured model, where FIG. 13A shows a plot of AB fiber, FIG. 13B shows a plot of C fiber, FIG. 13C shows a plot of A peak time, and FIG. 13D shows a plot of area under the curve.

FIG. 14 shows an experimental set up for future closed-loop peripheral nerve stimulation.

FIG. 15 shows a closed-loop implantable neurostimulator implanted in a lower back of a patient, and in particular, implanted to provide for spinal cord stimulation, according to examples of the present disclosure.

FIG. 16 shows a closed-loop implantable neurostimulator implanted in a lower back of a patient, and in particular, implanted to provide for peripheral nerve stimulation by interacting with the sciatic nerve, according to examples of the present disclosure.

FIG. 17 shows a computer-implemented method for controlling an implantable neurostimulator system for mitigating chronic pain according to examples of the present disclosure.

FIG. 18 is an example of a hardware configuration for a computer device according to examples of the present disclosure.

FIG. 19 shows a comparison of the Linear Parameter Varying (LPV) model and a Linear Time-Invariant (LTI) model according to examples of the present disclosure.

FIG. 20A and FIG. 20B show an example of how to set up a model with structured uncertainty (FIG. 20A) and to identify a controller, K, using μ synthesis (FIG. 20B) according to examples of the present disclosure.

FIG. 21A, FIG. 21B, FIG. 21C, and FIG. 21D show examples of a model with structured uncertainty to capture the nonlinearity in recorded naïve (FIG. 21A and FIG. 21B) and injured rat (FIG. 21C and FIG. 21D) electrophysiology data according to examples of the present disclosure.

FIG. 22A and FIG. 22B show a generalized block diagram of the closed-loop system with the VPL Thalamus response as the feedback to the PNS controllers according to examples of the present disclosure.

FIG. 23 shows an example of the experimental recording setup to simultaneously record WDR neuron spiking activity and VPL thalamus local field potentials in response to peripheral nerve stimulation according to examples of the present disclosure.

FIG. 24 is similar to FIG. 16 and shows a closed-loop implantable neurostimulator implanted in a lower back of a patient, and in particular, using feedback from the VPL Thalamus to provide peripheral nerve stimulation by interacting with the sciatic nerve, according to examples of the present disclosure.

It should be noted that some details of the figures have been simplified and are drawn to facilitate understanding of the present teachings rather than to maintain strict structural accuracy, detail, and scale.

### DETAILED DESCRIPTION

In examples of the present disclosure, the limitations of the conventional approaches are addressed by building a computational framework for a novel adaptive, model-based closed-loop peripheral nerve stimulation approach for the restoration of the dysfunctional pain system back to a healthy state. Generally speaking, examples of the present disclosure provides for construction of a linear mathematical model of the spinal cord using electrophysiology recordings from rats. Based on experiments, accurate predictions of the neural responses to electrical stimulation of the peripheral nerve are developed. Also, control systems techniques are applied to drive the dynamics of the chronic pain model into normal ranges by using closed-loop control of the PNS. This computational framework will guide the development of new closed-loop PNS therapies for chronic pain and improve treatment for one of the most prevalent diseases on earth.

Critical to advancing chronic pain treatment is a deeper understanding of pain transmission and the effects of neuromodulation under both normal and pathological conditions. However, these answers remain unclear because the pain system is complex and utilizes tightly regulated dynamical crosstalk between the peripheral nervous system and the brain via the spinal cord.

As an example, suppose a splinter pricks your toe. The body's initial response starts where the splinter stimulated a nerve fiber, which is part of a nociceptor, or pain sensor. The activated nociceptor produces an electrical impulse that travels up the leg to the spinal cord, which contains a cluster of cells called the dorsal horn. Within the dorsal horn, the impulse is processed and then sent up to the thalamus in the brain. Next, the thalamus sends the information that your toe has been pricked to the somatosensory cortex (which senses it), the frontal cortex (which thinks about it), and the limbic system (which reacts to it emotionally). However, these simple steps only scratch the surface of the perplexing complexity of pain. For example, make the splinter injury worse—a broken toe. The fracture hurts because the tissue and nerves around the damaged bone have suffered trauma. However, eight weeks later, the bone and nerves have mended, but the pain can still persist.

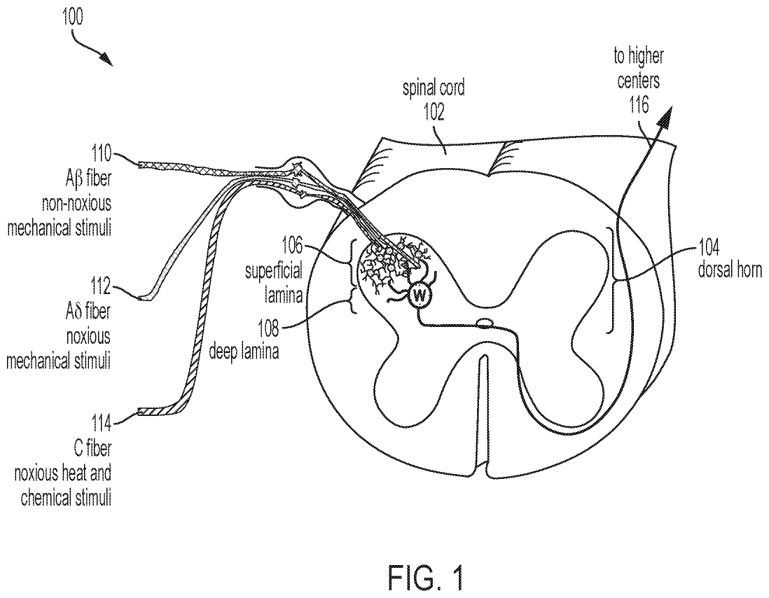

It is not clear why pain can continue after healing and this remains an open question because the pain system is difficult to probe (experimental barriers) and difficult to analyze (computational barriers). FIG. 1 shows an illustration 100 of the dorsal horn 104 in the spinal cord 102. Afferent fibers including the Aβ fiber 110, the Aδ fiber 112, and the C fiber 114, enter the superficial lamina 106 and the WDR neuron (W) in the deep lamina 108 projects the impulse to higher centers 116, such as the brain. The dorsal horn of the spinal cord, as shown in FIG. 1, is the first central relay station for sensory inputs. The high-threshold peripheral afferent fibers (Aδ and C) mainly send noxious information and terminate at superficial lamina, whereas the low-threshold peripheral afferent fibers (Aβ) primarily transmit innocuous inputs and terminate in the deeper lamina. In the superficial lamina, the inhibitory and excitatory interneurons create a modulatory circuit, which can regulate ascending pain transmission. The deep lamina consists of convergent neurons, or wide-dynamic range (WDR) neurons, because they receive both noxious and innocuous inputs. Many WDR neurons are projection neurons that are also involved in ascending pain transmission.

Analyzing electrophysiological recordings of these neurons in the dorsal horn can provide important dynamic information about changes in response due to injury, disease, drug treatments, or neuromodulation for chronic pain. Importantly, previous studies have linked pain perception to the firing patterns of WDR neurons. For example, “wind up” is an increase in pain perception to repetitive noxious stimulation, which is also observed in the progressively increased response of the WDR neurons. WDR windup behavior can be modulated by opioids, SCS, and PNS. However, it is difficult to study the physiological responses of dorsal horn neurons while simultaneously differentiating them. Therefore, the neuron's identity is unknown prior to the recording because tracing and staining of injected dye is done after the experiment.

A complement to conducting difficult biological experiments is to build mathematical models of the dorsal horn circuit. In 1965, Melzack and Wall proposed “gate-control theory” of pain which is the first static model of pain modulation in the dorsal horn. The model describes how the dorsal horn inhibitory interneurons act as a functional gate that “opens” or “closes”, which results in relaying or blocking pain transmission to the brain, respectively. The development of this model was timely as it showed the need for such models, however, it fails to explain the dynamics of certain firing patterns (e.g. wind up) and the relationships between these patterns and pain conditions. Since then, various detailed conductance-based dynamical models and finite element models have been built to describe: i) the different dorsal horn neurons and their interconnections; ii) the effects that SCS has on the dorsal horn circuit; and, iii) the effects of PNS on nerve fiber activation. These models can reproduce some of the observed behaviors, but they assume a fixed circuit topology, are high-dimensional, and nonlinear. They are not amenable to analysis because analytically characterizing a set of sensory stimuli, model parameters, and treatment parameters that produce the observed firing patterns is nearly impossible.

Currently, chronic pain is primarily treated with drugs, which may be inadequate or harmful, have significant negative side effects (e.g., opioid addiction), and can lose efficacy after long-term use. Alternatively, chronic pain is also treated with neuromodulation approaches to target the spinal cord (SCS) or the peripheral nerves (PNS). A comprehensive list of stimulation devices for both SCS and PNS can be found in [30]. Patients using neuromodulation experience a higher quality of life and greater pain relief relative to individuals using drugs. However, the choice of stimulation parameters, including pulse duration and frequency, can have considerable effects on the clinical outcomes. Currently, only around half of the patients reported successful outcomes, which is over a 50% reduction in pain. Neuromodulation pain therapies have been associated with suboptimal efficacy and limited long-term success as their mechanisms of action are unclear. Also, nearly all neuromodulation therapies are open-loop, which means that the parameters of the stimulation (i.e. amplitude, frequency, pulse width) must be manually adjusted.

In order to fully capture the neuromodulation needs of the user, the next step in advancing neuromodulation therapies is to “close the loop” and use feedback to adjust the stimulation in real-time. The objective is to use closed-loop stimulation to reduce the amplified pain signals caused by injury or disease, while still maintaining normal pain processing capabilities of the dorsal horn. It is theorized that if the dorsal horn dynamics of the system experiencing chronic pain are made to match a healthy response, then the brain will only perceive normal responses, thereby reducing chronic pain levels. However, significant challenges emerge when closing the loop, including, for example, what controller and feedback signal to use to modulate the stimulation.

Currently, only one experimental study has shown the advantage of closed-loop neuromodulation in humans, specifically SCS. In this study, the authors implemented a closed-loop SCS system to automatically adjust the stimulation to maintain a constant number of activated fibers in the dorsal column, as the participant changes position. The feedback signal chosen is the evoked compound action potentials.

Additionally, a closed-loop experiment focused on inhibiting nociceptive signals in rats showed positive results. The firing rate of the WDR neurons is continually monitored and initiates the electrical neurostimulation if the firing rate reaches a set threshold. The electrical neurostimulation is applied to the periaqueductal gray, in the brain. Once the WDR firing rate reached dips below the threshold, then the neurostimulation stops.

Mathematical models of pain transmission are disclosed that are used to inform the development of closed-loop neuromodulation treatments, specifically PNS. A tractable computational model of the dorsal horn pain-processing circuit is developed that is consistent with electrophysiological data that are used to analyze and test how sensory stimuli and PNS modulate pain perception. A cellular switch of the projection neurons is theorized as responsible for the short-term functional modulation of pain transmission in the dorsal horn. Projection neurons are not faithful followers of sensory neurons action potentials trains and display complex membrane properties that transform their inputs. This model characterizes the dynamical balance of intrinsic parameters and exogenous inputs that drives this switch. A primary advantage of the model is that it is computationally efficient, low-dimensional, and able to capture the nature of neuronal dynamics in the dorsal horn. By applying linear control systems techniques, the PNS is controlled so as to drive the dynamics of the chronic pain dorsal horn model and a chronic pain VPL Thalamus model to mimic the healthy dorsal horn and VPL Thalamus model dynamics. Therefore, closed-loop PNS therapies can automatically adapt based on the patient's current need to reduce pain transmission to the brain.

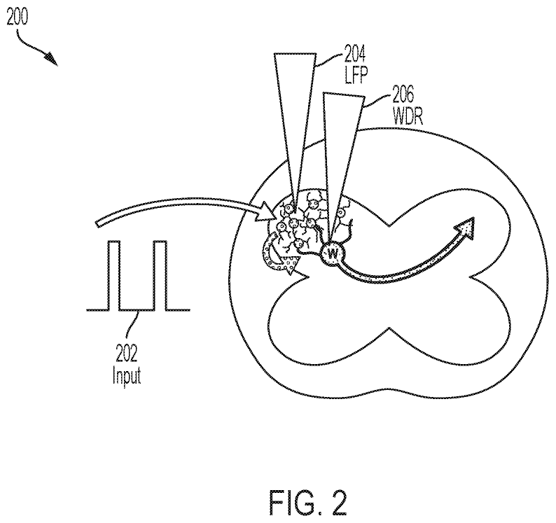

Electrophysiological measurements are recorded in vivo from three adult male rats. All procedures are approved by the Johns Hopkins University Animal Care and Use Committee. FIG. 2 shows a biophysical representation 200 of the dorsal horn where the black triangles represent the recording microelectrodes and the WDR neuron (W) projects the impulse to the brain. Fine-tip microelectrodes 204 and 206 are used to measure neuronal activity in the superficial lamina 106 and the deep lamina 108 of the dorsal horn 104, which correspond to the local field potential (LFP) and spiking activity from the WDR neurons, respectively. The sampling frequency of the dataset is 10,000 Hz. To perturb the dorsal horn, a paired-pulse input 202 is applied to the peripheral sciatic nerve. Each pulse is 5 mA, biphasic, and 0.5 ms long and there is a 400 ms delay between the 1^{st} and 2^{nd} pulse. In total, ten trials are recorded for each animal. Due to the difficulties of simultaneously measuring from various subsets of dorsal horn neurons, the LFP and WDR responses are recorded individually. Thus, each of the trials is time-locked to the first pulse input for model construction.

The experiments are performed under two sets of conditions, naive and injured. The first condition is where the rat is healthy, which will be referred to as the naive condition. The second condition is where the rat has been given a spinal cord injury and experiences chronic pain and will be referred to as the injured condition. For the dataset described herein, a full set of LFP and WDR recordings are recorded for the naive animal. Due to experimental limitations, for the injured condition, one rat is used to record the LFP responses and another rat to record the WDR spiking activity.

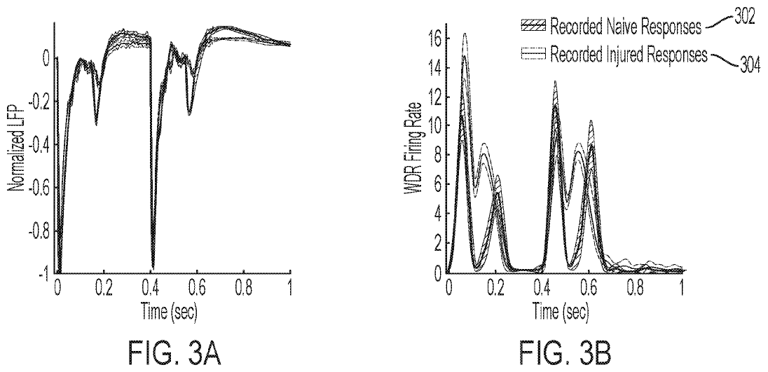

The initial preprocessing step for the superficial lamina LFP data is to downsample the timeseries to 1,000 Hz by using the interp1 function in Matlab. Then, to reduce high-frequency oscillations and artifacts due to heartbeat and noise, the smooth function in Matlab is applied to the data. The function smooths the timeseries by using an N-point moving average, where N is chosen to be 100. Next, the processed timeseries are normalized so that for both the injured and naive responses the minimum peak is equal to negative one, as shown in FIG. 3A. This normalization step is done to mitigate the amplitude effect of electrode depth across conditions.

FIG. 3A and FIG. 3B show a comparison of the naive 302 and nerve-injured 304 rat responses from the superficial lamina (LFP recordings) in FIG. 3A and deep lamina (WDR firing rate) in FIG. 3B. The solid line and shaded area indicate the mean and standard deviation over the ten trials, respectively. For the WDR neuron spiking recordings, the first step is to identify the spikes by thresholding the recorded timeseries. The threshold is set as four times the standard deviation of the recording before the stimulation starts. After thresholding, the timeseries becomes a series of ones and zeros, where a one indicates a spike at a particular time point and zero shows no spike. Next, to compute the firing rate, the binary spiking timeseries is convolved with a 1000-point Gaussian window to create the smooth firing rate shown in FIG. 3B. The Matlab functions used to convolve the data and create the Gaussian window are cony and gausswin, respectively. Finally, the firing rate curves are downsampled to 1,000 Hz to match LFP data.

The evoked spinal LFP responses, shown in FIG. 3A, corresponding to the activation of peripheral A- and C-fibers by electrical test stimuli can be distinguished on the basis of conduction velocity and activation threshold. Central terminals of C-afferent neurons transmit noxious information to the spinal cord, mostly in the superficial dorsal horn where C-LFP is recorded. Thus, C-LFP is a high-throughput approach for examining broad and net nociceptive transmission in the superficial dorsal horn in vivo. Compared to that in naive rats, the peak amplitude of C-LFP is decreased in nerve-injured rats. This may be due to injuries to both central terminals of C-afferent neurons and dorsal horn neurons after injury, which would decrease afferent C-fiber inputs as well as population response of superficial dorsal horn neurons.

Interestingly, WDR neurons response, shown in FIG. 3B, in deeper dorsal horn lamina shows an increased C-component response to electrical test stimulation. In addition, the interval between A-component and C-component that is seen in naive animals is shortened and less clear in nerve-injured rats. The reasons for this remain unclear, but it may be due to several reasons. The injury can induce complicated changes in spinal neuronal circuitry, such as a loss and decreased function of inhibitory interneurons. Thus, although afferent inputs may be decreased after injury, ascending pain signals transmitted by post-synaptic secondary neurons, such as WDR neurons, may increase due to increased excitability. Changes in the intervals between A- and C-component of WDR neurons may be partially due to changes in conduction properties of afferent fibers, altered synaptic transmission, and the development of spontaneous activity and after discharges of both primary sensory neurons and WDR neurons after injury.

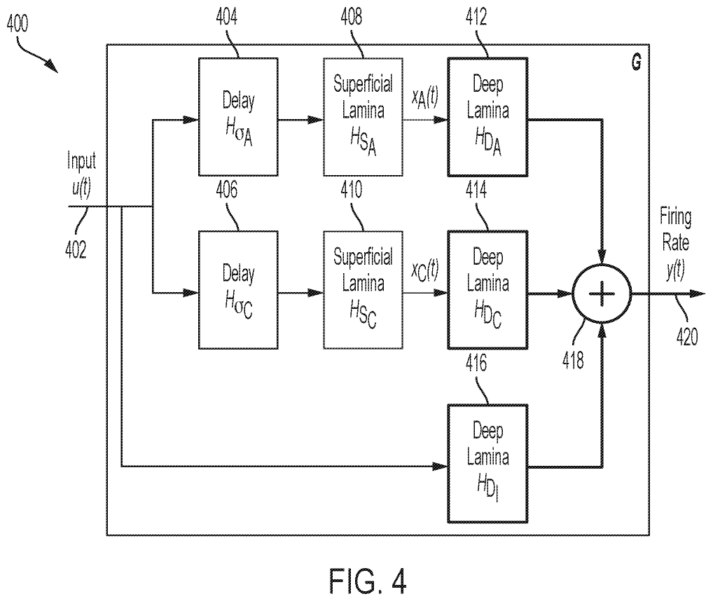

The objective of the proposed dorsal horn and VPL model is to predict the superficial and deep lamina responses and VPL Thalamus local field potential (LFP) activity from only the peripheral nerve stimulation. The LFP responses and WDR firing rates are modeled using linear time-invariant (LTI) continuous-time transfer functions. FIG. 4 shows the generalized block diagram 400 of the full model, which is identified for both the naive rat condition, G_{N}, and the injured rat condition, G_{I}. The full model, G, is made up of five different transfer functions. First, the input 402, u(t), is delayed by using the transfer functions (H_{σ}_{_{A}} 404 and H_{σ}_{_{c}} 406), which represent the transmission delays on the Aβ fiber and C fibers, respectively. Then, the respective delayed input responses are used as the input to the superficial transfer functions for Aβ fiber (H_{S}_{_{A}} 408) and C fibers (H_{S}_{_{C}} 410). Finally, the output of the LFP transfer functions (x_{A}(t) and x_{C}(t)) in addition to the initial input 402, u(t), are set as the inputs to the three transfer functions (H_{D}_{_{A}} 412, H_{D}_{_{C}}, 414, and H_{D}_{_{I}}, 416) at combiner 418 characterizing the WDR firing rate, y(t) 420, respectively.

For both the injured and naive rat responses, 80% of the data is used to train the models and 20% is used for validation. To identify the best fits for each transfer function, a search grid is utilized to optimize different parameters. For each condition, the final parameters, for all transfer functions, are chosen such that the root-mean-squared error (RMSE) is minimized over all parameter combinations.

In order to approximate the transmission delays, a lowpass Bessel filter [47] is implemented to delay the input pulse stimulation. For this application, a Bessel filter is ideal because it produces a flat delay up to the chosen angular cutoff frequency. In Matlab, to create the Bessel filter, the filter order, n, and angular cutoff frequency, Wₒ, must be defined. The resulting standard form for H_{σ}_{_{A}} and H_{σ}_{_{C}} is

$${H}_{σ} ( s ) = \frac{{α}_{0}}{{β}_{0} {s}^{n} + {β}_{1} {s}^{n - 1} + {β}_{2} {s}^{n - 2} + … + {β}_{n}} \qquad ( 1 )$$

where α0 and β0 through βn are coefficients fit using the besself Matlab function. For both H_{σ}_{_{A}} and H_{σ}_{_{c}} two parameters need to be chosen using a search grid. The first parameter is the Bessel filter order, n, which is set to be between 1 and 25. The second parameter is the angular cutoff frequency, Wo, which is limited to be between 100 rad/s and 2,000 rad/s. Transitioning to the next component of the model, the superficial lamina LFP transfer functions for the Aβ fiber and C fiber (H_{S}_{_{A}}) and C fibers (H_{S}_{_{A}}), can be described as

$$H ( s ) = \frac{{a}_{0} {s}^{z} + {a}_{1} {s}^{z - 1} + {a}_{2} {s}^{z - 2} + … + {a}_{z}}{{b}_{0} {s}^{p} + {b}_{1} {s}^{p - 1} + {b}_{2} {s}^{p - 2} + … + {b}_{p}} \qquad ( 2 )$$

where α₀ through α_{z}, and b₀ through bₚ are fit using the tfest Matlab command. For H_{S}_{_{A}} and H_{S}_{_{C}}, a search is performed to identify the optimal number of zeros and poles in the transfer function. The search grid is set so that the number of zeros, could be between 1 and 4, and the number of poles could be between 2 and 5 with the condition that z<p.

To identify the best transfer functions for both the transmission delays and superficial lamina, the grid search is combined. Overall, the parameter search included 6 terms, two for the Aβ component (n, Wo) and two for the C component (n, Wo), and the number of zeros, z, and poles, p, which are constrained to be the same for both H_{S}_{_{A}} 408 and H_{SC} 410. The summed output of x_{A}(t) and x_{C}(t) is compared to the validation LFP dataset. For each condition (naive and injured), the final parameters are chosen such that the RMSE is minimized over all parameter combinations.

The next step in building the full model is to fit the three transfer functions to predict the WDR firing rate response. As shown in FIG. 4, the inputs to the deep lamina transfer functions (H_{D}_{_{A}} 412, H_{D}_{_{C}}, 414, and H_{D}_{_{I}}416) are x_{A}(t), x_{C}(t) and, u(t) 402, respectively. The outputs of all three transfer functions are summed to compute the WDR firing rate response, y(t) 420.

Similar to before, a search is performed to identify the optimal number of zeros and poles in the three deep lamina transfer functions. The search grid is set so that the number of zeros can be between 1 and 19, and the number of poles can be between 2 and 20 with the condition that z<p. Similar to the superficial lamina transfer functions, the number of poles and zeros, which are constrained to be identical in the H_{D}_{_{A}} 412 and H_{D}_{_{C}}, 414 transfer functions.

For each parameter combination, the coefficients of the transfer function listed in Equation (2) are fit using the tfest Matlab command. Like before, the RMSE is computed between the validation firing rate responses and the predicted firing rate response. For each condition, the final parameters are chosen such that the RMSE is minimized over all parameter combinations.

After optimizing the parameters, the full transfer function model, G, from FIG. 4 can have a high model order. The current full models are ideal to estimate each component of the response. The WDR firing rate of the injured model, G_{I}, is driven to mimic the responses observed in the naive model, G_{N}. Therefore, the full model can be simplified by applying balanced truncation [48] to the entire system by using the balred function in Matlab. The model is reduced to have 30 zeros and 31 poles, which maintains the desired low-frequency response.

The main goal is to develop a closed-loop controller that can drive the dynamics of the nerve-injured response to mimic the response observed in the healthy rats. Therefore, by using the reduced injured and naive models, H∞ model-matching is used to achieve the desired closed-loop performance [49, 50, 51, 52, 53].

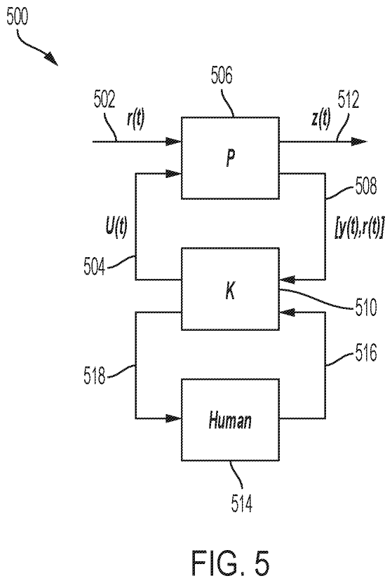

FIG. 5 shows a representation 500 of the standard H∞ optimization problem. In order to find an H∞ controller for this system, first, the problem must be expressed as an optimization problem using the standard configuration shown in FIG. 5. The two inputs to the plant 506, P, are the reference signal, r(t) 502, the controller output, U(t) 504. The output of the plant (such as the dorsal horn) 506 includes, z(t) 512, which are the error signals to be minimized and, r(t) 502, the reference signal, and the measured output, y(t). The controller, K 510, is determined during the optimization process. Due to the uncertainty and nonlinearity of the actual system, a multiplicative uncertainty (Human 514) is incorporated into the optimization process. The input to the controller, K, is the reference r(t) 502, the reference signal, and the measured output, y(t) 508 and is modulated by the uncertainty loop 516 518.

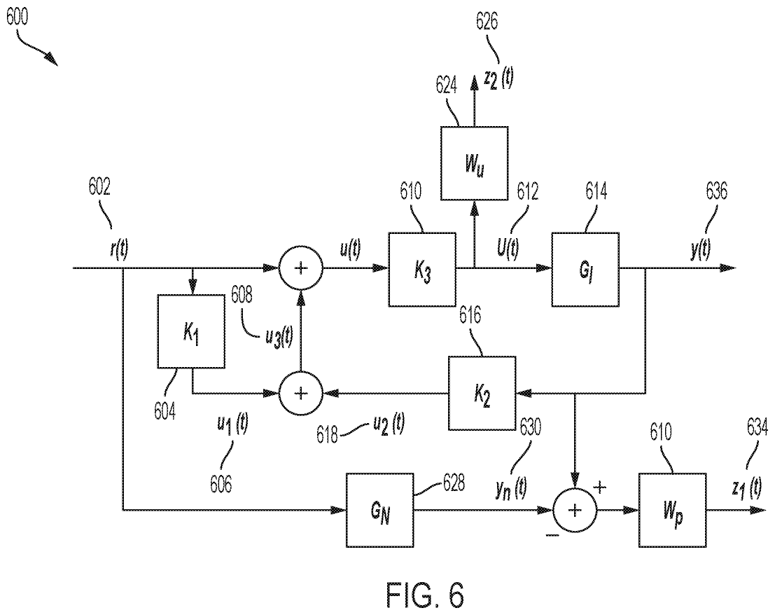

FIG. 6 shows the closed-loop system 600 using H∞ model-matching control system using the three controllers K₁, K₂, and, K₃ 606, identified during the optimization process to drive the injured model, G_{I} 614, to match the naive model, G_{N} 618. Wᵤ and Wₚ are frequency-dependent weighting functions to ensure desired performance. Wᵤ 610 acts to limit the high-frequency components of the controller output. Wₚ 622 ensures the closed-loop injured model and the naive model responses are close in the low-frequency range. The open-loop system, called P in FIG. 5, is described by

$$[ \begin{matrix}{z}_{1} ( t ) \\ {z}_{2} ( t ) \\ r ( t ) \\ y ( t )\end{matrix} ] = [ \begin{matrix}- {W}_{p} {G}_{N} & {W}_{p} {G}_{I} \\ 0 & {W}_{u} \\ I & 0 \\ 0 & - {G}_{I}\end{matrix} ] [ \begin{matrix}r ( t ) \\ U ( t )\end{matrix} ]$$

In addition, it can be shown that the closed-loop system is

$$[ \begin{matrix}{z}_{1} ( t ) \\ {z}_{2} ( t )\end{matrix} ] = Tr ( t ) \qquad ( 4 )$$

$$T = {[ \begin{matrix}{T}_{1} & {T}_{2}\end{matrix} ]}^{T} \qquad ( 5 )$$

$$and$$

$$\begin{matrix}{T}_{1} = {W}_{p} ( \frac{{K}_{3} ( {K}_{1} + 1 ) {G}_{I}}{1 - {K}_{2} {K}_{3} {G}_{I}} - {G}_{N} ) \\ {T}_{2} = {W}_{u} ( \frac{{K}_{3} ( {K}_{1} + 1 )}{1 - {K}_{2} {K}_{3} {G}_{I}} )\end{matrix} \qquad ( 6 )$$

The measured output, y (t), the desired naive model output, yₙ(t), and the control action, U(t), can be written as

$$y ( t ) = {G}_{I} U ( t ) \\ {y}_{n} ( t ) = {G}_{N} r ( t ) \\ U ( t ) = \frac{{K}_{3} ( {K}_{1} + 1 )}{1 - {K}_{2} {K}_{3} {G}_{I}} r ( t ) \qquad ( 7 )$$

The objective of the optimization problem shown in FIG. 5 is to find a controller, K, that minimizes the H∞ norm of T (Equation 5).

$${min}_{K stabilizing} {‖ \begin{matrix}{W}_{p} ( {K}_{3} ( {K}_{1} + 1 ) {{G}_{I} ( 1 - {K}_{2} {K}_{3} {G}_{I} )}^{- 1} - {G}_{N} ) \\ {W}_{u} ( {K}_{3} ( {K}_{1} + 1 ) {( 1 - {K}_{2} {K}_{3} {G}_{I} )}^{- 1} )\end{matrix} ‖}_{∞} \qquad ( 8 )$$

The first function minimized (T₁) is the weighted difference between the naive model, G_{N}, and the injured closed-loop system model, G_{I}. The second function minimized (T₂) is the weighted control action. In addition, Wₚ ensures the loop gain from r(t) to the error (y(t)-yₙ(t)) to be within a particular tolerance, tol. For this controller, Wₚ and Wᵤ are chosen to be

$$\begin{matrix}{W}_{p} = {Γ}_{p} \frac{2 s + 1}{2 s + tol} \\ {W}_{u} = {Γ}_{u} \frac{0.05 s + 1}{0.0001 s + 0.01}\end{matrix} \qquad ( 9 )$$

where Γₚ=0.01, tol=0.01, and Γᵤ=0.001. The parameters, Γₚ, tol, and Γᵤ, are chosen after doing a greedy search.

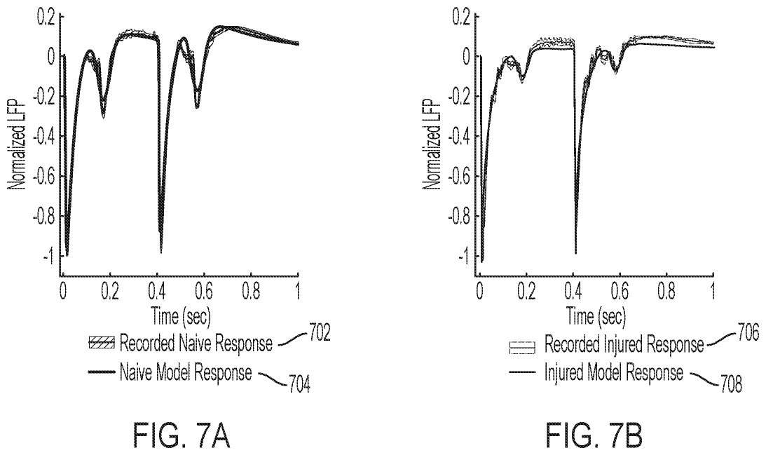

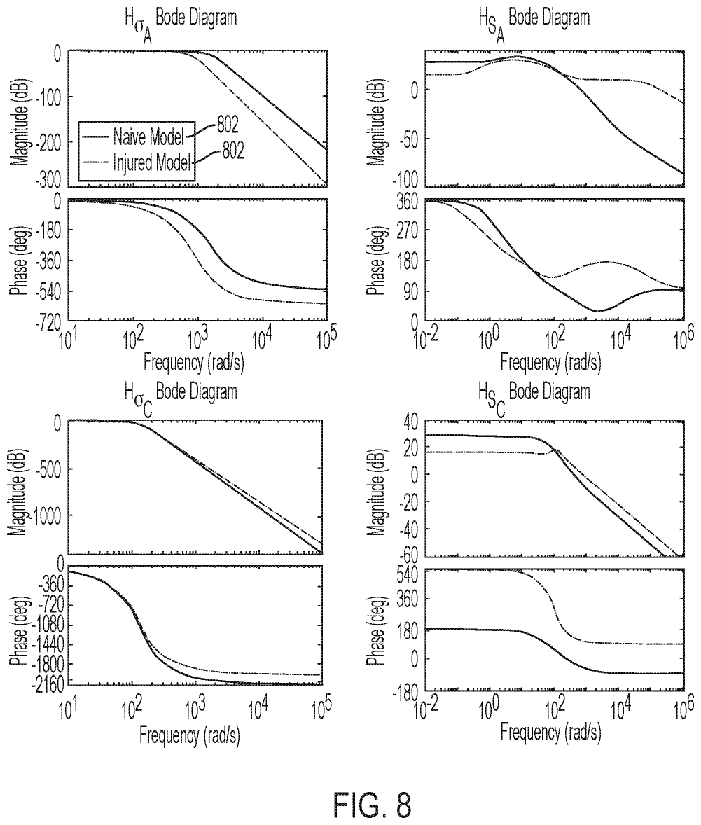

The Bessel filters (Ho) combined with a 3^{rd} order LTI continuous-time transfer functions (HS) can accurately capture the dynamics of the superficial lamina in response to the paired-pulse input. FIG. 7A and FIG. 7B show a comparison of the recorded and estimated LFP responses from the optimized transfer functions for the naive and nerve-injured rats, respectively. FIG. 7A shows a plot of the recorded naïve response 702 and the naïve model response 704 and FIG. 7B shows a plot of the recorded injured response 706 and the injured model response 708. Table 1 lists the optimal values for the Bessel delay function and the model order for the superficial lamina transfer functions chosen from the search grid. The Bessel delay function is defined by the order, n, and the angular cutoff frequency, Wo. The superficial lamina LFP transfer function is defined by the number of poles, p, and zeros, z. For the naive model, the RMSE between the recorded and estimated LFP responses is 0.0136 and 0.0143 for training and testing, respectively. For the injured model, the RMSE between the recorded and estimated LFP responses is 0.017 and 0.0162 for training and testing, respectively. FIG. 8 shows a comparison of the bode diagrams of the delay and superficial LFP transfer functions for naive 802 and injured 804 models. Therefore, using an LTI transfer function can quickly and accurately predict the LFP response to a paired-pulse input in both the naive and injured conditions.

TABLE 1
The optimal parameters chosen from the grid search for the
Bessel delay and the superficial lamina LFP transfer functions.

| | H_{σA} | | H_{σC} | | H_{AS}, H_{SC} | |
|---|---|---|---|---|---|---|
| | Order | Frequency | Order | Frequency | Zeros | Poles |
| Condition | (n) | (Wₒ) | (n) | (Wₒ) | (z) | (p) |
| Naïve | 6 | 1500 | 24 | 128 | 2 | 3 |
| Injured | 7 | 780 | 22 | 120 | 2 | 3 |

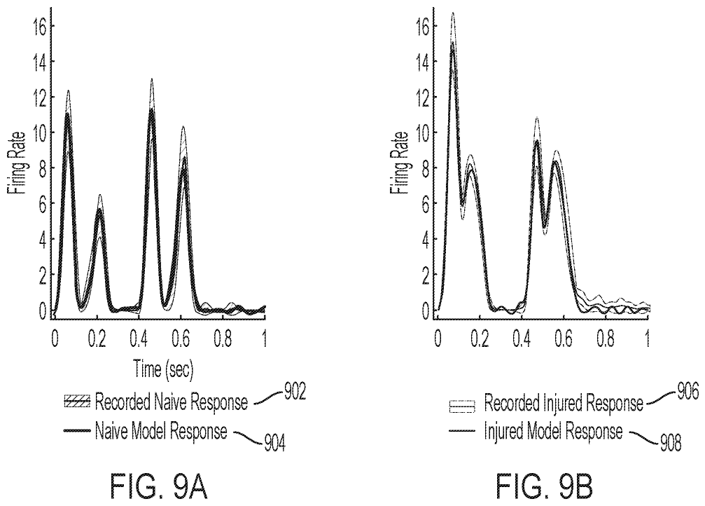

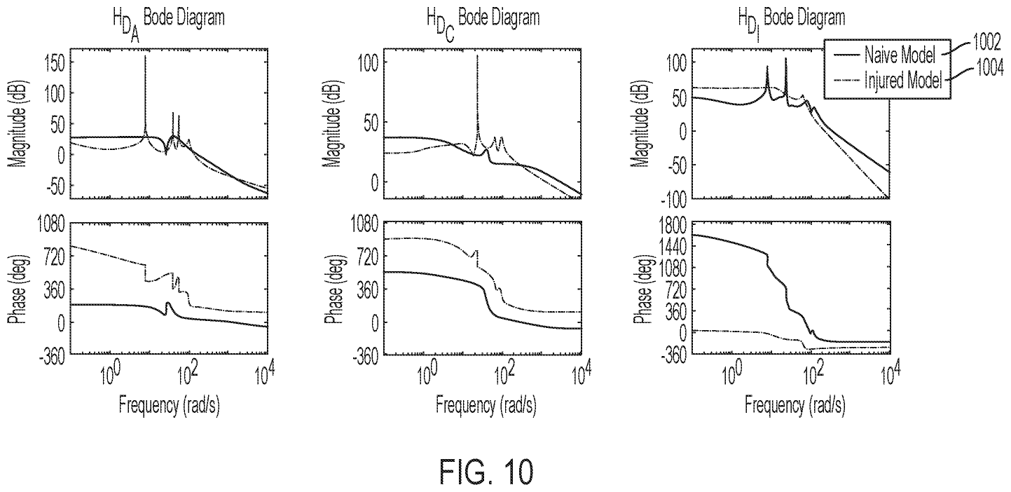

In addition, the deep lamina WDR firing rate response can be captured using LTI continuous-time transfer functions. FIGS. 9A and 9B show a comparison of the firing rates from the recorded WDR activity and the predicted model output, where FIG. 9A shows the recorded naive response 902 and the naïve model response 904 and FIG. 9B shows the recorded injured response 906 and the injured model response 908. Table 2 lists the optimal number of poles, p, and zeros, z, for the three transfer functions defining the WDR activity. For the naive model, the RMSE between the recorded and estimated WDR responses are 0.3012 and 0.2772 for training and testing, respectively. For the injured rat, the RMSE between the recorded and estimated LFP responses are 0.2530 and 0.2529 for training and testing, respectively. FIG. 10 shows a comparison of the bode diagrams of the deep lamina WDR transfer functions for both the naive model 1002 and the injured model 1004. Therefore, the WDR firing rate response can accurately be predicted using an LTI transfer function for both the naive and injured conditions.

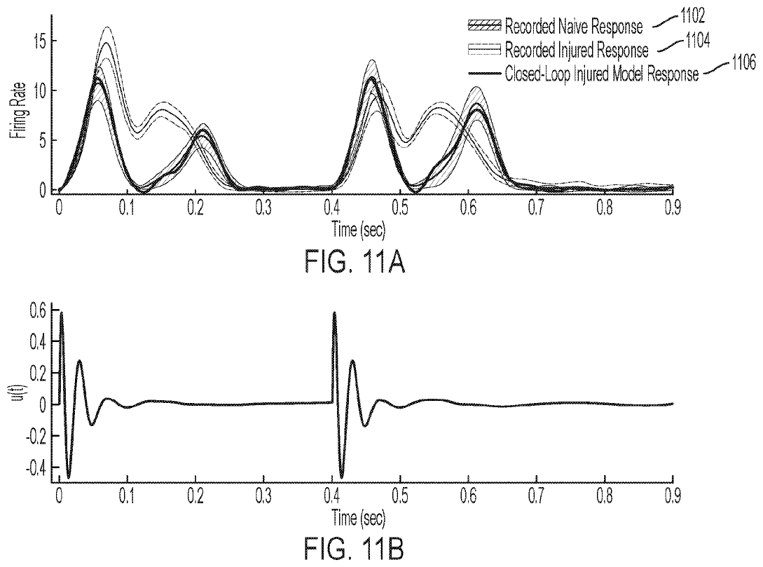

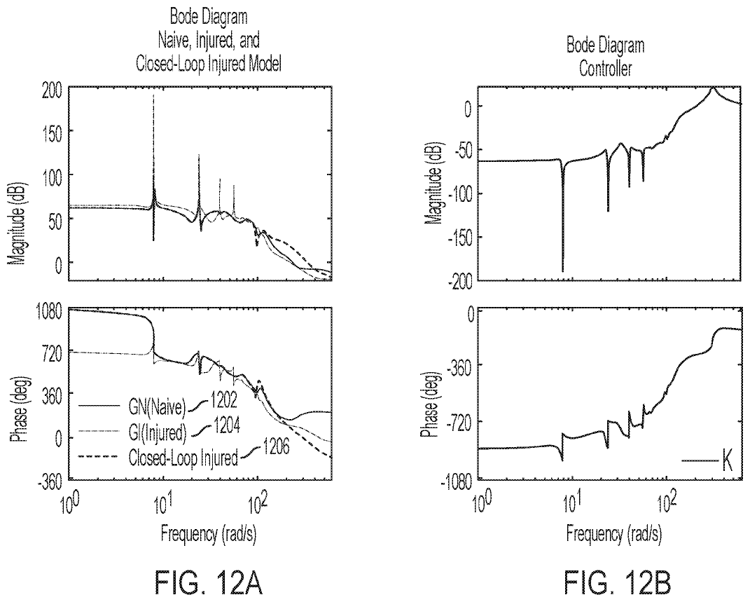

H∞ model-matching control can successfully drive the dynamics of the injured rat model to mimic the dynamics of the naive model. FIG. 11A shows the result of the closed-loop injured model response (line 1106) overlaid on the recorded WDR firing rate responses for the naive (line 1102) and injured (line 1104) rats. The objective of the controller is to drive the red line to match the blue. Overall, the controller is achieving an RMSE tracking error of 0.228. FIG. 11B shows the resulting controller output, u(t), which acts as the input to the injured model. The bode diagrams for the naïve 1202, injured 1204, and closed-loop injured 1206 model responses are shown in FIG. 12A. The closed-loop injured model is driven to overlay the naive model nearly perfectly in both magnitude and phase until approximately 100 rad/sec. The frequency of the paired-pulse stimulation (approximately 16 rad/sec) is well below the 100 rad/sec deviation point. FIG. 12B shows the bode diagram for the controller, K, described in FIG. 6.

TABLE 2
The optimal parameters chosen from the grid search for
deep lamina WDR transfer functions.

| | H_{AD}, H_{DC} | | H_{DI} | |
|---|---|---|---|---|
| Condition | Zeros (z) | Poles (p) | Zeros (z) | Poles (p) |
| Naïve | 3 | 4 | 13 | 15 |
| Injured | 9 | 10 | 1 | 4 |

FIG. 9 shows a comparison of the recorded and estimated WDR firing rates from the optimized transfer function for the naive and nerve-injured rats.

FIG. 10 shows a comparison of the bode diagrams for the fitted deep lamina models for the naive and nerve-injured rats.

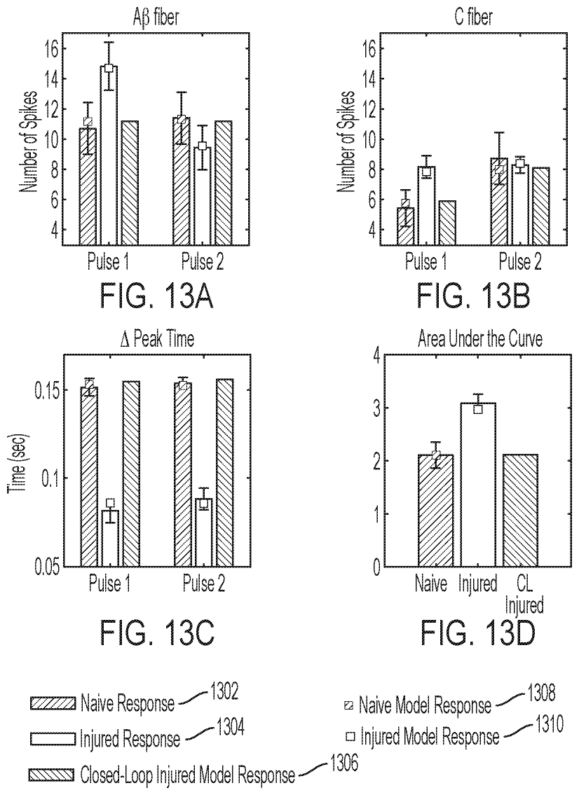

In order to compare the results of the closed-loop controller and the recorded WDR firing rate activity, a set of summary metrics are shown in FIGS. 13A-13D. FIG. 13A and FIG. 13B show a comparison of the number of spikes present in the AB and C fiber components of the WDR response for each pulse, respectively. The A peak time is a measure of the distance between the two peaks in the firing rate curves. Finally, the area under the curve metric is a measure of the area under the firing rate curves, which is found by integration. Bar 1302 and bar 1304 indicate the mean responses for the naive and injured rat recordings, respectively and the error lines are one standard deviation. Square 1308 on top of the naive and square 1310 on top of the injured responses show the predictions of the naive and injured models. Bar 1306 shows the predicted closed-loop injured model response. For each of the metrics, the naive and injured model responses are within one standard deviation of the recorded responses. In addition, and most importantly, the closed-loop injured model responses are restored to naive conditions in each of the metrics.

FIG. 13: A comparison of metrics describing the firing rate responses in the naive rat, injured rat, and closed-loop injured model.

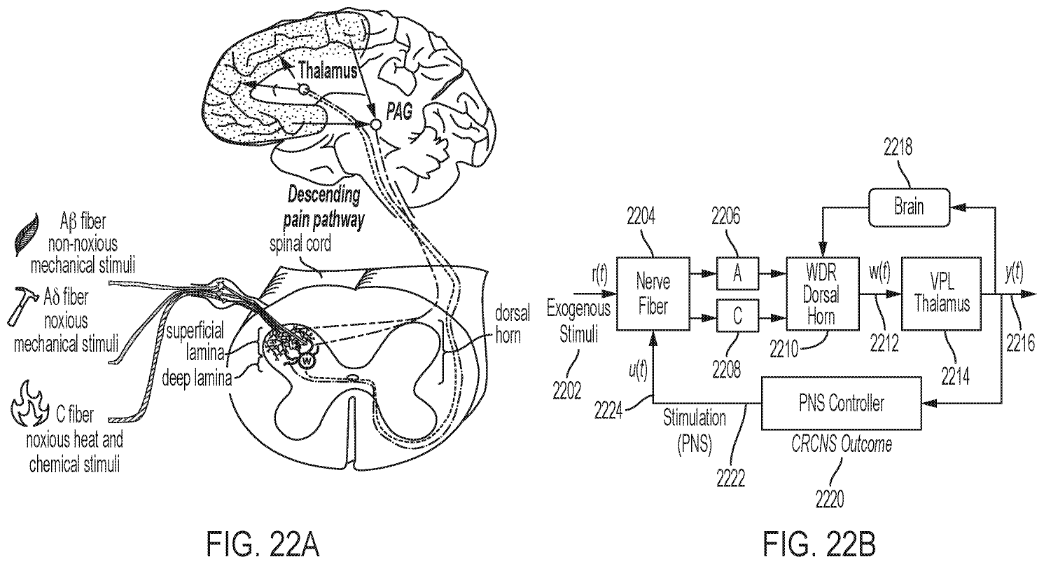

The previous example focused on the results of the paired-pulse stimulus which produced a fairly linear response. However, other inputs with varying amplitude and frequency can produce nonlinearities observed in the responses. One reason for the varying response is due to the complexity of the pain system (shown in FIG. 22A, which is an expansion of FIG. 1).

Now we show a different example of how to close the loop using recordings from the wide-dynamic range neurons (WDR) neurons in the dorsal horn of the spinal cord in vivo, a cell type selected for (i) its well documented deviation from its healthy baseline in pain syndromes making it a reliable physiological readout for pain, and (ii) role as the first central relay station between pain receptors in the periphery. Additionally, we will record from local field potential (LFP) which reflect activities of populations of neurons in the ventral posterolateral nucleus (VPL) of the thalamus, a gateway for pain information to enter the brain for perception. The thalamus can be accessed and recorded from using deep brain stimulation electrodes in humans, making it an ideal location for pain therapies designed for translation. VPL will thus be the feedback measurement used for the closed-loop PNS. See FIG. 22B for an example block diagram version that incorporates the PNS controller into the pain system. In this example, an exogenous stimuli r(t) 2202 travels down a nerve fiber 2204 and the activity is split into the non-noxious (A fiber 2206) and noxious (C fiber 2208) responses. Then the WDR activity in the dorsal horn 2210 is transmitted to VPL Thalamus 2214. Next, the response from VPL Thalamus is sent back to the dorsal horn to modulate ascending pain transmission via the brain 2218. Finally, the output of VPL Thalamus, y(t) 2216, is sent to the PNS controller 2220 which determines the PNS stimulation 2222 control signal u(t) 2224

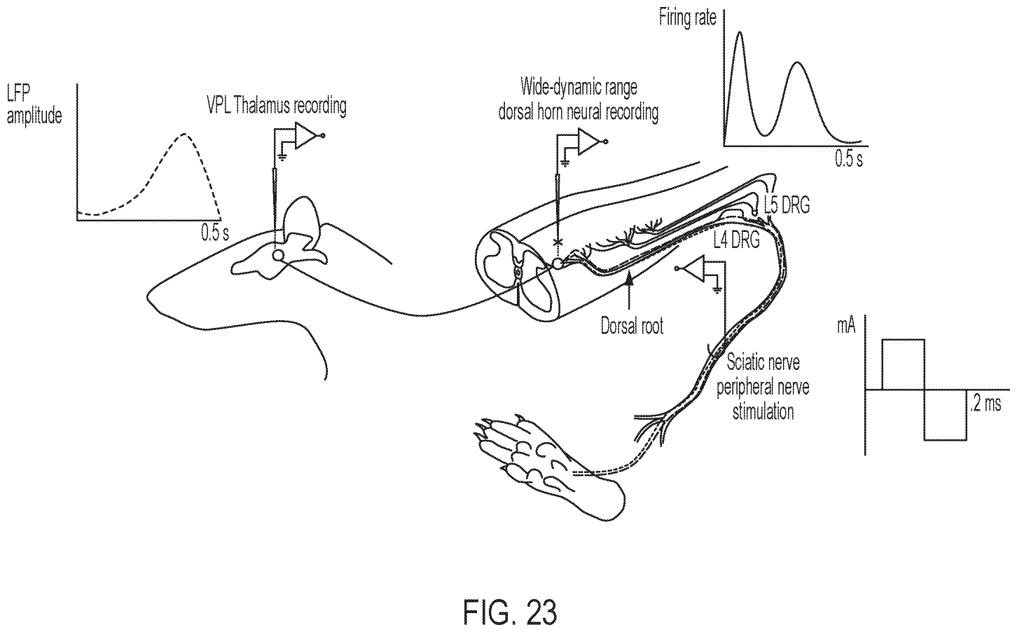

We follow similar steps to the previous example, where mathematical models will be built using an electrophysiological dataset. An example setup to simultaneously record from both WDR and VPL is shown in FIG. 23. Here the paw of the rat is mechanically or electrically stimulated to provide an exogenous stimulus. The therapeutic PNS is delivered to the sciatic nerve. Recordings are collected from the WDR (in the form of spiking activity) and VPL thalamus (in the form of local field potentials).

One way of modeling the WDR and VPL activity is through that linear parameter varying (LPV) model can capture the variations in the WDR PNS responses as opposed to a single LTI model. In the state-space realization of LPV models, a scheduling parameter determines the state-space matrices used to predict the WDR response. To develop the LPV models, the strictly proper LTI TFs (Eqn 2) are identified for each individual stimulation amplitude using their respective responses. This generated a set of LTI TF models, one for each stimulation amplitude, which are then transformed into state-space representations. Therefore, our final LPV model is created by using the set of LTI models and a scheduling parameter which determines the state-space model evaluated for that instant in time. We chose our scheduling parameter, p, to be the integral of current delivered over the last 20 seconds, effectively giving us the amplitude of the single pulse as our parameter but allowing for history effects due to more complex inputs in future work.

To evaluate and select a best-fit LPV model, a root-mean-squared error (RMSE) is calculated between the firing rate curve and model-predicted curve for every stimulation amplitude and every pole-zero combination. The average RMSE is then evaluated over all amplitude pulses. Further, the order of the model (number of poles) was penalized to find an ideal model that balances model complexity and accurate representation. This gave a single pole-zero combination to be used going forward. To evaluate the performance of the LPV model, we also identified a single LTI model of the same pole-zero combination to act as a reference.

For constructing the LPV model, an interpolation scheme is needed to estimate the response for any value of the scheduling parameter without an associated trained LTI model. Common methods include using the nearest neighbor (in scheduling parameter space) and linear interpolation. We chose a linear interpolation scheme, whereby the A, B, C, and D matrices of the state space model are linearly interpolated from the two nearest neighbor models. For this work, we utilized the interpolation scheme built into MATLAB's LPV Block in Simulink.

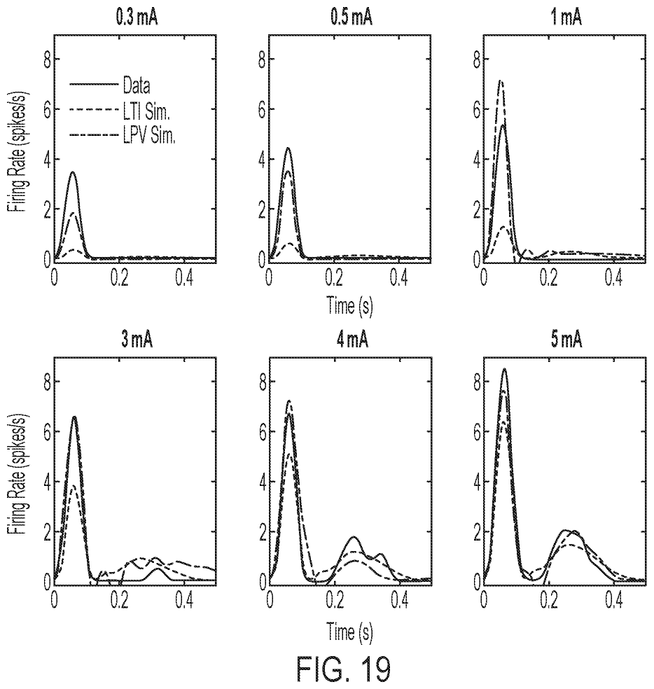

FIG. 19 shows simulated results for eighth order linear time invariant (LTI) and linear parameter varying (LPV) models. LPV results simulated using a two-nearest-neighbor linear interpolation scheme. Interpolation appears to perform best at small and high amplitudes and less well where the system transitions from one to two peaks according to examples of the present disclosure. As shown in FIG. 19, this LPV model is evaluated with a linear interpolation scheme by systematically leaving out each of our eight internal grid points (0.3-5 mA) and simulating the response using interpolation of the two nearest neighbors. For example, to find the interpolated model for 3 mA we interpolated between the state-space systems for 2 mA and 4 mA. The same inputs were simulated using the LTI model generated with equal poles and zeros. Two metrics were used to evaluate and compare the two model fits relative to the recorded firing rate data: R-squared (R2) coefficient of a linear fit and a root-mean-squared error (RMSE).

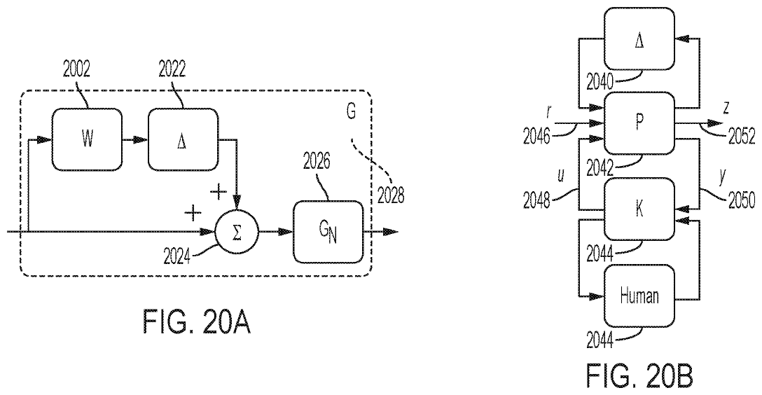

FIG. 20A and FIG. 20B show an example of how to set up a model with structured uncertainty (FIG. 20A) and to identify a controller, K 2004, using μ synthesis (FIG. 20B) according to examples of the present disclosure. Overall, it is shown that the nonlinear response can be accurately captured using an LPV model. The set of LTI models is transformed into a linear model with structured uncertainty. Generally, the structured uncertainty quantifies the amount of deviation (i.e. uncertainty) in response from the nominal (average) LTI model, across all inputs. For this application, the structured uncertainty is modeled in the input multiplicative form (FIG. 20A), where the model G 2028 is defined as the following set of transfer functions:

G=G_{N}(I+WΔ)

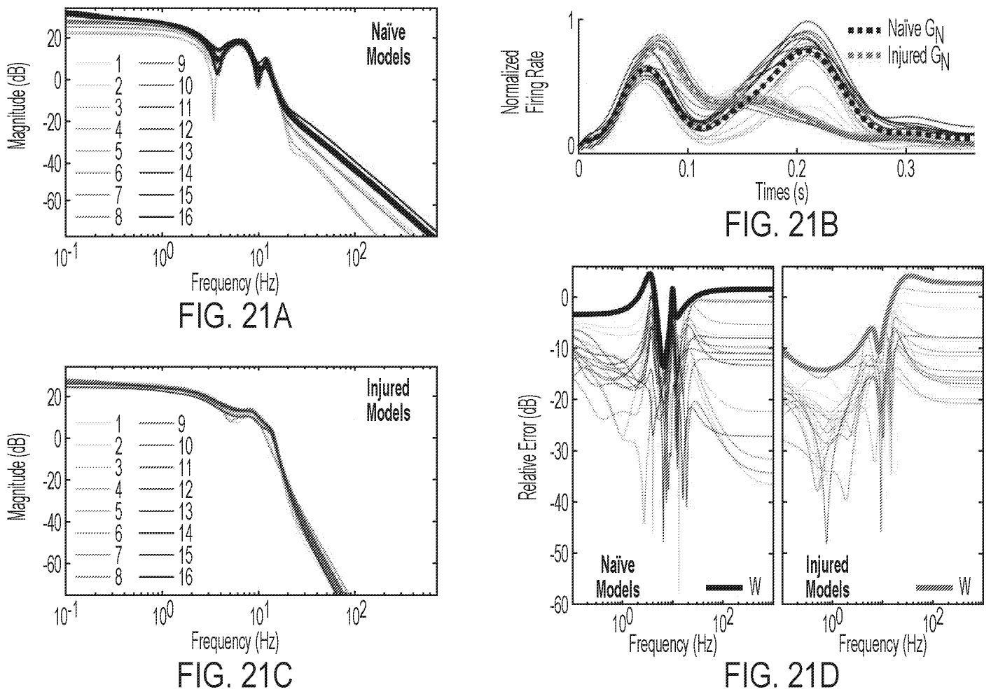

The nominal system (G_{N} 2026) is defined to be the average TF over all inputs. FIG. 21A, FIG. 21B, FIG. 21C, and FIG. 21D show examples of a model with structured uncertainty to capture the nonlinearity in recorded naïve (FIG. 21A and FIG. 21B) and injured rat (FIG. 21C and FIG. 21D) electrophysiology data according to examples of the present disclosure. In particular, FIG. 21A and FIG. 21B show the nominal responses (thick dotted lines) compared to the set of input responses (thin lines), for the naive and injured conditions. An additional TF (Δ 2022 and 2040) represents the uncertain dynamics with a unit peak gain. Finally, W 2002 is a fitted stable Nth order minimum-phase weighting function whose magnitude is greater than the largest relative error (between G_{N} and each model in the set). Therefore, W contains the amount of uncertainty in the potential windup responses, at all frequencies. The input to the nominal model 2026 is the sum 2024 of the input and the output of the W 2002 and Δ 2022 transer functions. For example, if the high frequency dynamics are not captured by the nominal model, then the weighting function acts as a high pass filter to reflect the frequencies with the largest amount uncertainty.

Overall, the variation (i.e. uncertainty) in the set of naive models is greater than in the set of injured model. We can quantify the uncertainty by computing the relative error between the nominal model and each of the individual models in the set. Referencing the block diagram in FIG. 20B, a frequency-dependent uncertainty weighting function, W, is fitted by using the maximum relative error at each frequency, over the set of LTI systems shown in FIG. 21A and FIG. 21C, for both conditions. For this application, a 5th order weighting function was chosen. In FIG. 21D, the dark thick lines indicate the weighting function, W, and the thin lines show the relative error between the nominal models and each of the LTI models in the sets.

By finding where the relative error and the uncertainty weighting functions is greater than 0 dB, the range of frequencies where the variation is largest across all models in the set and the specific windup pulse models that produce the largest variation for a particular frequency. For example, in the naive model, the greatest amount of variation, across all sixteen windup pulses, is between 2 Hz-5 Hz and 8 Hz-11 Hz. Additionally, it is found that pulse 1 and pulse 16 varied the most at 4 Hz and 10 Hz, respectively. Alternatively, for the injured rat, the largest variation in responses occurs in frequencies higher than 25 Hz, and the largest high frequency variations are observed in windup pulses 1, 3, and 8. Overall, using structured uncertainty is a useful method for exploring the underlying dynamics of the DH that cannot be identified using traditional methods.

Using the previously found structured uncertainty, a robust controller (K) is used for μ-synthesis, and rewriting the problem as an optimization problem using the Linear Fractional Transformation shown in FIG. 20B. The disclosed system, P, 2042 contains the nominal system, G_{N} 2026, the performance weighting functions, and the uncertainty weighting functions, W 2022. The A block 2022 and 2040 represents all the model uncertainty. The inputs to P are the reference signal, r 2046, the controller output, u 2048, and the uncertainty in the input. Additionally, the outputs of P 2042 are z 2052, which are the error signals that will be minimized, the measured output, y 2050, and the uncertainty in the output. An additional multiplicative uncertainty (Human 2044) is included in the optimization process to identify the controller, K 2044.

In addition to using LPV and uncertain dynamics, Koopman embedding of nonlinear dynamics to linear dynamics can be used. In particular, nonlinear dynamics can be sparsely identified through dynamic mode decomposition and candidate nonlinear functions relating states of our system. This allows the use of many of the established control techniques for linear dynamics with the disclosed nonlinear system.

Additionally, to compare the results of the robust controllers, it can be tested against other types of linear and nonlinear controllers. For example, linear controllers comprise Proportional—Integral— Derivative (PID), Linear quadratic regulator (LQR), and fuzzy logic control. For the nonlinear controllers, examples comprise extremum seeking control, predictor-based adaptive output feedback control, model-predictor control, model reference adaptive control, model identification adaptive control, iterative learning control, gain scheduling, backstepping, and sliding mode control.

A tractable computational model of the dorsal horn responses is constructed for both healthy and nerve-injured rats based on LFP and WDR recordings obtained from the superficial lamina network and the deep lamina, respectively. Responses of the dorsal horn circuit to electrical stimulation of the peripheral sciatic nerve using LTI transfer function models can thus be accurately predicted. The model is computationally efficient, low-dimensional, and able to capture the nature of neuronal dynamics in the dorsal horn. In addition, the H∞ model-matching control is shown to able to drive responses of the injured model to match the desired responses of the healthy rat. By using closed-loop control of the PNS, the goal is to reduce the amplified pain signals caused by injury or disease, while still maintaining normal pain processing capabilities of the dorsal horn. The restoration of normal pain perception in the brain is theorized to be achieved if the dorsal horn dynamics of the chronic pain model is driven to resemble the healthy responses.

In a simulation environment, achieving small closed-loop tracking error can be relatively straightforward. However, when implementing this system experimentally will produce significant challenges. The controllers developed in simulation might not easily translate to closed-loop experiments. Therefore, an iterative approach may be needed to optimize the controllers for in vivo implementation. The first step is to apply the resulting simulated closed-loop control action (shown in FIG. 11B) as an open-loop stimulation to the nerve-injured rat and observe the responses. Ideally, the closed-loop control action will produce responses similar to healthy rat control. Depending on the experimental set-up, the control action, U(t), may need to be discretized into a set of pulses. Collected new data can be integrated into the model, which can then be used to inform new experimental data collection.

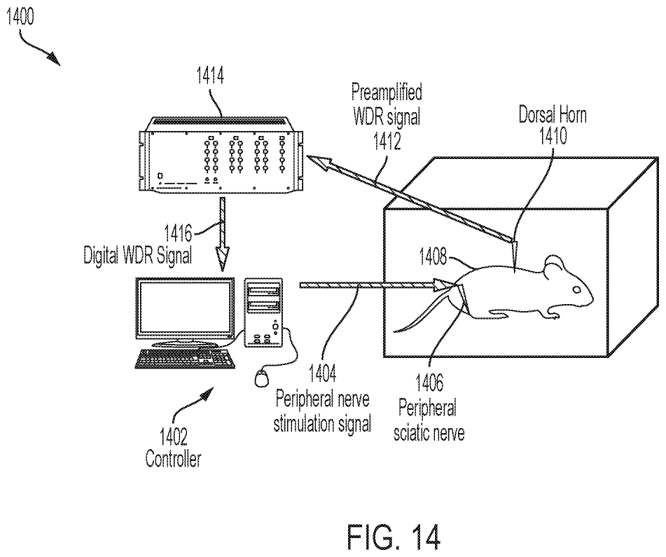

FIG. 14 shows a system for closed-loop peripheral nerve stimulation that automatically adjusts the peripheral nerve stimulation based on different exogenous stimuli, according to the present disclosure. First controller 1402 provides a peripheral nerve stimulation signal 1404 to peripheral sciatic nerve 1406 of a patient 1408. A preamplified WDR signal 1412 is detected at the dorsal horn 1410 and provided to a second controller 1414, which processes the preamplified WDR signal 1412 and provides a digital WDR signal 1416 to the first controller 1402. The closed-loop peripheral nerve stimulation automatically adapts to the individual's needs over time.

FIG. 22A and FIG. 22B show a generalized block diagram of the closed-loop system with the VPL Thalamus response as the feedback to the PNS controllers according to examples of the present disclosure. In particular, FIG. 22A shows the pain system, including ascending and descending pain pathways. Pain and touch information from the periphery converge on WDR neurons in the spinal cord. These signals are transmitted to the brain for further processing. FIG. 22B shows a block diagram of pain system shows an example of using a closed-loop PNS feedback controller with VPL as a feedback signal.

FIG. 23 show a data collection setup to simultaneously stimulate and record along the ascending pain pathway. PNS is delivered on the sciatic nerve. Then, the signal is transmitted to WDR neurons in the dorsal horn where the firing rate response is recorded. Then Pain information is sent to the thalamus where the local field response is recorded. WDR neurons in the spinal cord and VPL thalamus.

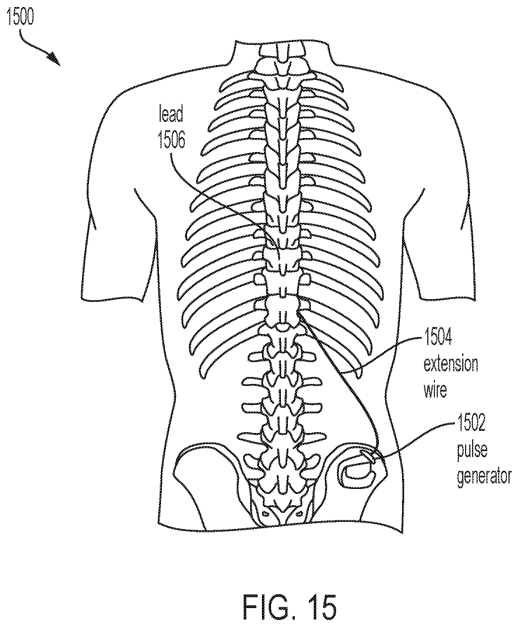

FIG. 15 shows an implantable neurostimulator 1500 implanted in a lower back of a patient, and in particular, implanted to provide for spinal cord stimulation, according to examples of the present disclosure. The lower back is just one example of a locations that the neurostimulator 1500 can be implanted. Other areas of the patient can be used for implantation depending of the particular individual being treated and the type of injury and location being treated. Closed-loop implantable neurostimulator 1500 comprises probe generator 1502 that includes a computer-readable medium and a controller configured to performed operations stored in the computer-readable medium, including the trained computer model as described herein. Probe generator 1502 is connected to an injury location using extension wire 1504 and lead 1506 to provide electrical impulses, described herein.

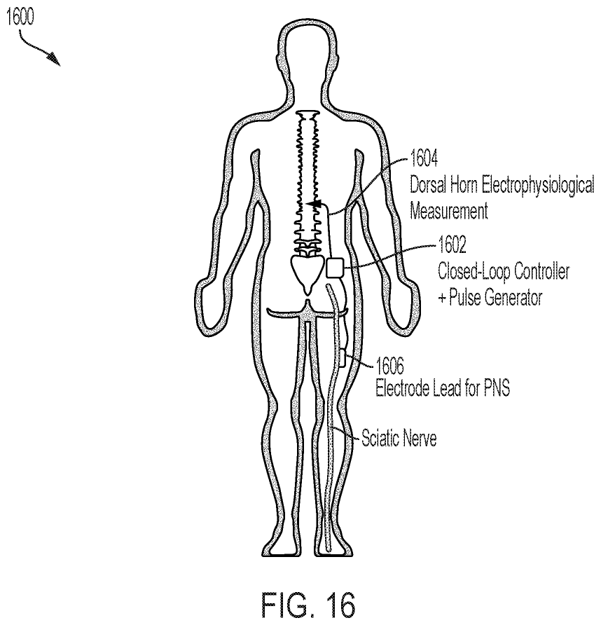

FIG. 16 shows an implantable neurostimulator 1600 implanted in a lower back of a patient, and in particular, implanted to provide for peripheral nerve stimulation by interacting with the sciatic nerve, according to examples of the present disclosure. Closed-loop implantable neurostimulator 1600 comprises probe generator 1602 that includes a computer-readable medium and a controller configured to performed operations stored in the computer-readable medium, including the trained computer model as described herein. Probe generator 1602 is connected to a dorsal horn to detect and/or provide electrical signals using extension wire 1604 and lead 1606 to provide electrical impulses to the sciatic nerve, described herein.

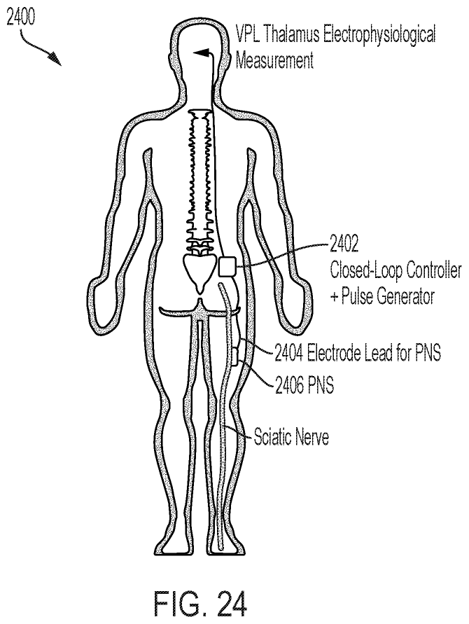

FIG. 24 is similar to FIG. 16 and shows an implantable neurostimulator implanted in a lower back of a patient, and in particular, implanted to provide for peripheral nerve stimulation by interacting with the sciatic nerve, according to examples of the present disclosure. Closed-loop implantable neurostimulator 2400 comprises probe generator 2402 that includes a computer-readable medium and a controller configured to performed operations stored in the computer-readable medium, including the trained computer model as described herein. Probe generator 2402 is connected to a VPL thalamus to detect and/or provide electrical signals using extension wire 2404 and lead 2406 to provide electrical impulses to the sciatic nerve, described herein.

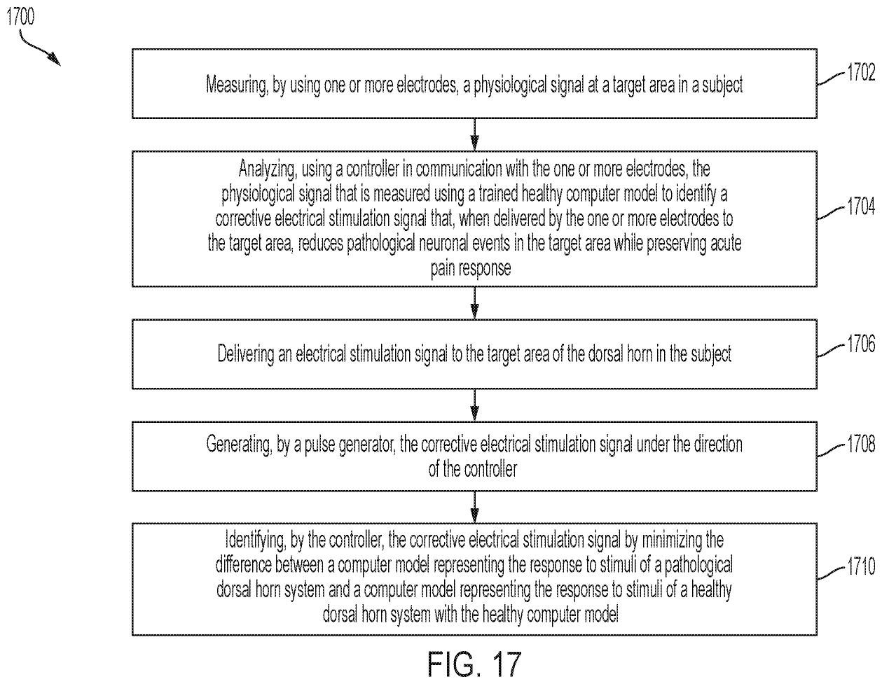

FIG. 17 shows a computer-implemented method 1700 for controlling an implantable neurostimulator system for mitigating chronic pain, according to examples of the present disclosure. The computer-implemented method 1700 comprises measuring, by using one or more electrodes, a physiological signal at a target area in a subject, as in 1702. In some examples, the target area comprises a dorsal horn, ventral posterolateral nucleus (VPL} Thalamus, peripheral nerves, or combinations of the dorsal horn, VPL Thalamus, and peripheral nerves. In some examples, the target area comprises a superficial lamina of the dorsal horn, a deep lamina of the dorsal horn, VPL Thalamus, peripheral nerves, or combinations of the superficial lamina of the dorsal horn, the deep lamina of the dorsal horn, the VPL Thalamus, the peripheral nerves.

The computer-implemented method 1700 continues by analyzing, using a controller in communication with the one or more electrodes, the physiological signal that is measured using a trained healthy computer model to identify a corrective electrical stimulation signal that, when delivered by the one or more electrodes to the target area, reduces pathological neuronal events in the target area while preserving acute pain response, as in 1704. In some examples, the corrective electrical stimulation signal causes at least one of neuronal inhibition in the target area, neuronal excitation in the target area, and no induced change to neuronal activity in the target area.

The computer-implemented method 1700 continues by delivering an electrical stimulation signal to the target area of the dorsal horn in the subject, as in 1706.

In some examples, the computer-implemented method 1700 further comprises generating, by a pulse generator, the corrective electrical stimulation signal under the direction of the controller, as in 1708.

In some examples, the computer-implemented method 1700 further comprises identifying, by the controller, the corrective electrical stimulation signal by minimizing the difference between a computer model representing the response to stimuli of a pathological dorsal horn system and a computer model representing the response to stimuli of a healthy dorsal horn system with the healthy computer model, as in 1710.

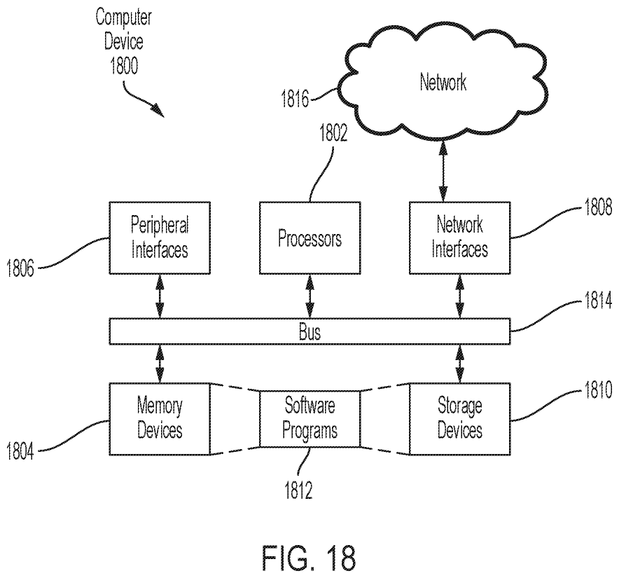

FIG. 18 is an example of a hardware configuration for a computer device 1800, which can be used to perform one or more of the processes described above. The computer device 1800 can be any type of computer devices, such as desktops, laptops, servers, etc., or mobile devices, such as smart telephones, tablet computers, cellular telephones, personal digital assistants, etc. As illustrated in FIG. 18, the computer device 1800 can include one or more processors 1802 of varying core configurations and clock frequencies. The computer device 1800 can also include one or more memory devices 1804 that serve as a main memory during the operation of the computer device 1800. For example, during operation, a copy of the software that supports the above-described operations can be stored in the one or more memory devices 1804. The computer device 1800 can also include one or more peripheral interfaces 1806, such as keyboards, mice, touchpads, computer screens, touchscreens, etc., for enabling human interaction with and manipulation of the computer device 1800.

The computer device 1800 can also include one or more network interfaces 1808 for communicating via one or more networks, such as Ethernet adapters, wireless transceivers, or serial network components, for communicating over wired or wireless media using protocols. The computer device 1800 can also include one or more storage devices 1810 of varying physical dimensions and storage capacities, such as flash drives, hard drives, random access memory, etc., for storing data, such as images, files, and program instructions for execution by the one or more processors 1802.

Additionally, the computer device 1800 can include one or more software programs 1812 that enable the functionality described above. The one or more software programs 1812 can include instructions that cause the one or more processors 1802 to perform the processes, functions, and operations described herein, for example, with respect to the process of FIG. 17. Copies of the one or more software programs 1812 can be stored in the one or more memory devices 1804 and/or on in the one or more storage devices 1810. Likewise, the data utilized by one or more software programs 1812 can be stored in the one or more memory devices 1804 and/or on in the one or more storage devices 1810. Peripheral interface 1806, one or more processors 1802, network interfaces 1808, one or more memory devices 1804, one or more software programs, and one or more storage devices 1810 communicate over bus 1814.

In implementations, the computer device 1800 can communicate with other devices via a network 1816. The other devices can be any types of devices as described above. The network 1816 can be any type of network, such as a local area network, a wide-area network, a virtual private network, the Internet, an intranet, an extranet, a public switched telephone network, an infrared network, a wireless network, and any combination thereof. The network 1816 can support communications using any of a variety of commercially-available protocols, such as TCP/IP, UDP, OSI, FTP, UPnP, NFS, CIFS, AppleTalk, and the like. The network 1816 can be, for example, a local area network, a wide-area network, a virtual private network, the Internet, an intranet, an extranet, a public switched telephone network, an infrared network, a wireless network, and any combination thereof.

The computer device 1800 can include a variety of data stores and other memory and storage media as discussed above. These can reside in a variety of locations, such as on a storage medium local to (and/or resident in) one or more of the computers or remote from any or all of the computers across the network. In some implementations, information can reside in a storage-area network (“SAN”) familiar to those skilled in the art. Similarly, any necessary files for performing the functions attributed to the computers, servers, or other network devices may be stored locally and/or remotely, as appropriate.

In implementations, the components of the computer device 1800 as described above need not be enclosed within a single enclosure or even located in close proximity to one another. Those skilled in the art will appreciate that the above-described componentry are examples only, as the computer device 1800 can include any type of hardware componentry, including any necessary accompanying firmware or software, for performing the disclosed implementations. The computer device 1800 can also be implemented in part or in whole by electronic circuit components or processors, such as application-specific integrated circuits (ASICs) or field-programmable gate arrays (FPGAs).

If implemented in software, the functions can be stored on or transmitted over a computer-readable medium as one or more instructions or code. Computer-readable media includes both tangible, non-transitory computer storage media and communication media including any medium that facilitates transfer of a computer program from one place to another. A storage media can be any available tangible, non-transitory media that can be accessed by a computer. By way of example, and not limitation, such tangible, non-transitory computer-readable media can comprise RAM, ROM, flash memory, EEPROM, CD-ROM or other optical disk storage, magnetic disk storage or other magnetic storage devices, or any other medium that can be used to carry or store desired program code in the form of instructions or data structures and that can be accessed by a computer. Disk and disc, as used herein, includes CD, laser disc, optical disc, DVD, floppy disk and Blu-ray disc where disks usually reproduce data magnetically, while discs reproduce data optically with lasers. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Combinations of the above should also be included within the scope of computer-readable media.

The foregoing description is illustrative, and variations in configuration and implementation can occur to persons skilled in the art. For instance, the various illustrative logics, logical blocks, modules, and circuits described in connection with the embodiments disclosed herein can be implemented or performed with a general purpose processor, a digital signal processor (DSP), an application specific integrated circuit (ASIC), a field programmable gate array (FPGA), cryptographic co-processor, or other programmable logic device, discrete gate or transistor logic, discrete hardware components, or any combination thereof designed to perform the functions described herein. A general-purpose processor can be a microprocessor, but, in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

In one or more exemplary embodiments, the functions described can be implemented in hardware, software, firmware, or any combination thereof. For a software implementation, the techniques described herein can be implemented with modules (e.g., procedures, functions, subprograms, programs, routines, subroutines, modules, software packages, classes, and so on) that perform the functions described herein. A module can be coupled to another module or a hardware circuit by passing and/or receiving information, data, arguments, parameters, or memory contents. Information, arguments, parameters, data, or the like can be passed, forwarded, or transmitted using any suitable means including memory sharing, message passing, token passing, network transmission, and the like. The software codes can be stored in memory units and executed by processors. The memory unit can be implemented within the processor or external to the processor, in which case it can be communicatively coupled to the processor via various means as is known in the art.

What is claimed is:

1. A closed-loop implantable neurostimulator system for mitigating chronic pain, the closed-loop implantable neurostimulator system comprising:
a neuromodulation device comprising one or more electrodes configured to measure a physiological signal of a subject and deliver an electrical stimulation signal to a target area in the subject; and
a controller, in communication with the one or more electrodes, comprising a processor and a computer-readable memory storing a trained healthy computer model, the controller configured to analyze the physiological signal that is measured using the trained healthy computer model to identify a corrective electrical stimulation signal that, when delivered by the one or more electrodes to the target area, reduces pathological neuronal events in the target area while preserving acute pain response.

2. The closed-loop implantable neurostimulator system of claim 1, wherein the target area comprises a dorsal horn, ventral posterolateral nucleus (VPL) Thalamus, peripheral nerves, or combinations of the dorsal horn, VPL Thalamus, and peripheral nerves.

3. The closed-loop implantable neurostimulator system of claim 2, wherein the target area comprises a superficial lamina of the dorsal horn, a deep lamina of the dorsal horn, VPL Thalamus, peripheral nerves, or combinations of the superficial lamina of the dorsal horn, the deep lamina of the dorsal horn, the VPL Thalamus, the peripheral nerves.

4. The closed-loop implantable neurostimulator system of claim 1, wherein the corrective electrical stimulation signal causes at least one of neuronal inhibition in the target area, neuronal excitation in the target area, and no induced change to neuronal activity in the target area.

5. The closed-loop implantable neurostimulator system of claim 1, further comprises:
a pulse generator that generates the corrective electrical stimulation signal under direction of the controller.

6. The closed-loop implantable neurostimulator system of claim 4, further comprises:
a power source that powers the controller and a pulse generator.

7. The closed-loop implantable neurostimulator system of claim 1, further comprises:
an insulated lead coupling of one or more electrodes to the controller.

8. The closed-loop implantable neurostimulator system of claim 1, wherein the controller further identifies the corrective electrical stimulation signal by minimizing a difference between a computer model representing the response to stimuli of a pathological dorsal horn and/or VPL Thalamus system and a computer model representing the response to stimuli of a healthy dorsal horn and/or VPL Thalamus system.

9. A computer-implemented method for controlling an implantable neurostimulator system for mitigating chronic pain, the computer-implemented method comprising:
measuring, by using one or more electrodes, a physiological signal at a target area in a subject;
analyzing, using a controller in communication with the one or more electrodes, the physiological signal that is measured using a trained healthy computer model to identify a corrective electrical stimulation signal that, when delivered by the one or more electrodes to the target area, reduces pathological neuronal events in the target area while preserving acute pain response; and
delivering an electrical stimulation signal to the target area of a dorsal horn in the subject.

10. The computer-implemented method of claim 9, wherein the target area comprises a dorsal horn, ventral posterolateral nucleus (VPL} Thalamus, peripheral nerves, or combinations of the dorsal horn, VPL Thalamus, and peripheral nerves.

11. The computer-implemented method of claim 10, wherein the target area comprises a superficial lamina of the dorsal horn, a deep lamina of the dorsal horn, VPL Thalamus, peripheral nerves, or combinations of the superficial lamina of the dorsal horn, the deep lamina of the dorsal horn, the VPL Thalamus, the peripheral nerves.

12. The computer-implemented method of claim 9, wherein the corrective electrical stimulation signal causes at least one of neuronal inhibition in the target area, neuronal excitation in the target area, and no induced change to neuronal activity in the target area.

13. The computer-implemented method of claim 9, further comprises:
generating, by a pulse generator, the corrective electrical stimulation signal under direction of the controller.

14. The computer-implemented method of claim 9, further comprising identifying, by the controller, the corrective electrical stimulation signal by minimizing a difference between a computer model representing the response to stimuli of a pathological dorsal horn and/or VPL Thalamus system and a computer model representing the response to stimuli of a healthy dorsal horn and/or VPL Thalamus system with the healthy computer model.

15. A closed-loop implantable neurostimulator system for mitigating chronic pain, the closed-loop implantable neurostimulator system comprising:
a controller, in communication with one or more electrodes, comprising a processor and a computer-readable memory storing a trained healthy computer model, the controller configured to analyze a physiological signal that is measured using the trained healthy computer model to identify a corrective electrical stimulation signal that, when delivered by the one or more electrodes to a target area, reduces pathological neuronal events in the target area while preserving acute pain response.

16. The closed-loop implantable neurostimulator system of claim 15, wherein the target area comprises a dorsal horn, ventral posterolateral nucleus (VPL} Thalamus, peripheral nerves, or combinations of the dorsal horn, VPL Thalamus, and peripheral nerves.

17. The closed-loop implantable neurostimulator system of claim 16, wherein the target area comprises a superficial lamina of the dorsal horn, a deep lamina of the dorsal horn, VPL Thalamus, peripheral nerves, or combinations of the superficial lamina of the dorsal horn, the deep lamina of the dorsal horn, the VPL Thalamus, the peripheral nerves.

18. The closed-loop implantable neurostimulator system of claim 15, wherein the corrective electrical stimulation signal causes at least one of neuronal inhibition in the target area, neuronal excitation in the target area, and no induced change to neuronal activity in the target area.
